(19) United States
(12) Patent Application Publication
(10) Pub. No.: US 2026/0114744 A1
(43) Pub. Date: Apr. 30, 2026

(54) BIOLOGICAL INFORMATION DETECTION DEVICE

(71) Applicant: TERUMO KABUSHIKI KAISHA, Tokyo (JP)

(72) Inventor: Masaomi IMAI, Chuo-shi (JP)

(73) Assignee: TERUMO KABUSHIKI KAISHA, Tokyo (JP)

(21) Appl. No.: 19/003,218

(22) Filed: Dec. 27, 2024

Related U.S. Application Data
(63) Continuation of application No. PCT/JP2023/023623, filed on Jun. 26, 2023.

(30) Foreign Application Priority Data
Jun. 30, 2022 (JP) 2022-106684

(51) Int. Cl. A61B 5/053; A61B 5/00; A61B 5/0205; A61B 5/024

(52) U.S. Cl. CPC A61B 5/053; A61B 5/02055; A61B 5/02438; A61B 5/6824; A61B 5/6826; A61B 2560/0468; A61B 2562/043

(57) ABSTRACT

A biological information detection device that includes a main body including an arm placement surface on which a forearm or a wrist can be placed, and a finger support capable of supporting a finger of the user in a state in which the forearm or the wrist is placed on the arm placement surface, in which the main body includes a pair of arm electrodes exposed on the arm placement surface and capable of detecting the biological impedance from the forearm or the wrist, and in a top view of the biological information detection device as seen from a side of the arm placement surface, the finger support is attached to the main body so as to be movable in an approaching direction approaching the main body and a separating direction separating from the main body opposite to the approaching direction.

### CROSS-REFERENCES TO RELATED APPLICATIONS

This application is a continuation of International Application No. PCT/JP2023/023623 filed on Jun. 26, 2023, which claims priority to Japanese Application No. 2022-106684 filed on Jun. 30, 2022, the entire content of both of which is incorporated herein by reference.

### TECHNOLOGICAL FIELD

The present disclosure relates to a biological information detection device.

### BACKGROUND DISCUSSION

Conventionally, a biological information detection device that detects biological impedance by causing a current to flow from one hand or foot of a user to the other hand or foot via a torso is known. Japanese Patent Application Publication Nos. 2001-000408 A, 10-234690 A, 10-234692 A, and 2001-061805 A disclose this type of biological information detection device.

In the biological information detection device disclosed in Japanese Patent Application Publication Nos. 2001-000408 A, 10-234690 A, 10-234692 A, and 2001-061805 A, a current is caused to flow from one hand of a user to the other hand via a torso to detect biological impedance. Therefore, in a case where the user is a pacemaker implanted patient and the like, for example, the pacemaker might malfunction and cannot be used.

A wearable biological information detection device that is worn by being wound around an arm of a user and detects biological impedance from only one arm of the user is also conceivable, but there is still room for improvement from the viewpoint of detection accuracy due to positional displacement due to body movement, variation in a contact state due to a change in outer diameter of the arm due to edema, and an influence due to sweat.

### SUMMARY

A biological information detection device is disclosed, which capable of accurately detecting biological impedance from one arm of a user placed on the arm of the user.

A biological information detection device according a first aspect of the present disclosure is (1) a biological information detection device capable of detecting biological impedance from a forearm or a wrist of a user, the biological information detection device including: a main body including an arm placement surface on which the forearm or the wrist can be placed; and a finger support capable of supporting a finger of the user in a state in which the forearm or the wrist is placed on the arm placement surface, in which the main body includes a pair of arm electrodes exposed on the arm placement surface and capable of detecting the biological impedance from the forearm or the wrist, and in a top view of the biological information detection device as seen from a side of the arm placement surface, the finger support is attached to the main body so as to be movable in an approaching direction approaching the main body and a separating direction separating from the main body, the separating direction being opposite to the approaching direction.

The biological information detection device according to one embodiment of the present disclosure is (2) the biological information detection device according to (1) described above, in which in the top view, the pair of arm electrodes is arranged side by side in a direction substantially orthogonal to the approaching direction and the separating direction.

The biological information detection device as one embodiment of the present disclosure is (3) the biological information detection device according to (2) described above, in which the main body includes a pulse sensor capable of acquiring pulse information from the forearm or the wrist placed on the arm placement surface, and in a case where a direction in which the pair of arm electrodes is arranged in the top view is an electrode arrangement direction, the pulse sensor is arranged in a position between the pair of arm electrodes in the electrode arrangement direction in the top view.

The biological information detection device as one embodiment of the present disclosure is (4) the biological information detection device according to (2) or (3) described above, in which the main body includes an activation switch exposed on the arm placement surface, and in a case where the direction in which the pair of arm electrodes is arranged in the top view is the electrode arrangement direction, the activation switch is arranged in a position between the pair of arm electrodes in the electrode arrangement direction in the top view.

The biological information detection device as one embodiment of the present disclosure is (5) the biological information detection device according to any one of (1) to (4) described above, in which the main body includes a weight detection unit capable of detecting weight information of the forearm or the wrist placed on the arm placement surface.

The biological information detection device as one embodiment of the present disclosure is (6) the biological information detection device according to any one of (1) to (5) described above, in which the arm placement surface includes a circular arc groove extending from an end in the approaching direction to an end in the separating direction of the main body.

The biological information detection device as one embodiment of the present disclosure is (7) the biological information detection device according to any one of (1) to (6) described above, in which the finger support includes: a length adjustment unit movably attached to the main body in the approaching direction and the separating direction, the length adjustment unit being capable of adjusting a protruding length from the main body; and a finger placement table held by the length adjustment unit and including a finger placement surface on which an inner surface of the finger can be placed.

The biological information detection device as one embodiment of the present disclosure is (8) the biological information detection device according to (7) described above, in which one of the main body and the length adjustment unit includes a position fixing unit that fixes a relative position of the other of the main body and the length adjustment unit in the approaching direction and the separating direction.

The biological information detection device as one embodiment of the present disclosure is (9) the biological information detection device according to (7) or (8) described above, in which the finger placement surface includes a circular arc groove extending from an end in the approaching direction to an end in the separating direction of the finger placement table.

The biological information detection device as one embodiment of the present disclosure is (10) the biological information detection device according to (7) or (8) described above, in which the finger placement surface includes an inner surface of a through hole penetrating from an end in the approaching direction to an end in the separating direction of the finger placement table.

The biological information detection device as one embodiment of the present disclosure is (11) the biological information detection device according to any one of (7) to (10) described above, in which the finger placement table includes a pair of finger electrodes exposed on the finger placement surface and capable of detecting the biological impedance through the inner surface of the finger.

(12) A biological information detection device configured to detect biological impedance from a forearm or a wrist of a user according to another aspect includes: a main body including an arm placement surface on which the forearm or the wrist can be placed; a pair of arm electrodes exposed on the arm placement surface and configured to detect the biological impedance from the forearm or the wrist; a pulse sensor placed on the arm placement surface and configured to acquire pulse information from the forearm or the wrist; and wherein the pulse sensor is placed closer to a peripheral wall of the main body than the pair of arm electrodes so as to position the wrist of the user closer to the pulse sensor than the pair of arm electrodes when placing the forearm or the wrist of the user on the arm placement surface.

(13) A method for detecting biological impedance from a forearm or a wrist of a user according to a further aspect includes: receiving the forearm or the wrist of the user on an arm placement surface; supporting a finger of the user in a state in which the forearm or the wrist is placed on the arm placement surface, wherein in a top view as seen from a side of the arm placement surface, the finger support is attached to a main body so as to be movable in an approaching direction approaching and a separating direction, the separating direction being opposite to the approaching direction; and detecting the biological impedance from the forearm or the wrist with a pair of arm electrodes exposed on the arm placement surface.

According to the present disclosure, a biological information detection device capable of accurately detecting biological impedance from one arm of a user placed on the arm of the user.

### BRIEF DESCRIPTION OF THE DRAWINGS

FIG. 1 is a perspective view of a biological information detection device as one embodiment of the present disclosure.

FIG. 2 is a top view of the biological information detection device illustrated in FIG. 1.

FIG. 3 is a top view of the biological information detection device illustrated in FIG. 1, together with an arm and a hand of a user who uses the biological information detection device.

FIG. 4 is a diagram illustrating the inside of a main body of the biological information detection device illustrated in FIG. 1 in detail.

FIG. 5 is a perspective view of the biological information detection device illustrated in FIG. 1, the view illustrating a stored state of the biological information detection device.

FIG. 6 is a top view of the biological information detection device in the stored state illustrated in FIG. 5.

FIG. 7 is a diagram illustrating a variation of a finger placement surface of a finger placement table.

FIG. 8 is a diagram illustrating a variation of a finger placement surface of a finger placement table.

FIG. 9 is a diagram illustrating a variation of the biological information detection device illustrated in FIG. 1.

FIG. 10 is a diagram illustrating an accommodation case capable of accommodating the main body and a finger support illustrated in FIG. 1.

FIG. 11 is a diagram illustrating a variation of the biological information detection device illustrated in FIG. 1.

FIG. 12 is a diagram illustrating a variation of the biological information detection device illustrated in FIG. 1.

FIG. 13 is a diagram illustrating a variation of the biological information detection device illustrated in FIG. 1.

FIG. 14 is a diagram illustrating a variation of the biological information detection device illustrated in FIG. 1.

### DETAILED DESCRIPTION

An embodiment of a biological information detection device according to the present disclosure will be hereinafter described by way of example with reference to the drawings. In the drawings, the same components are denoted by the same reference numerals.

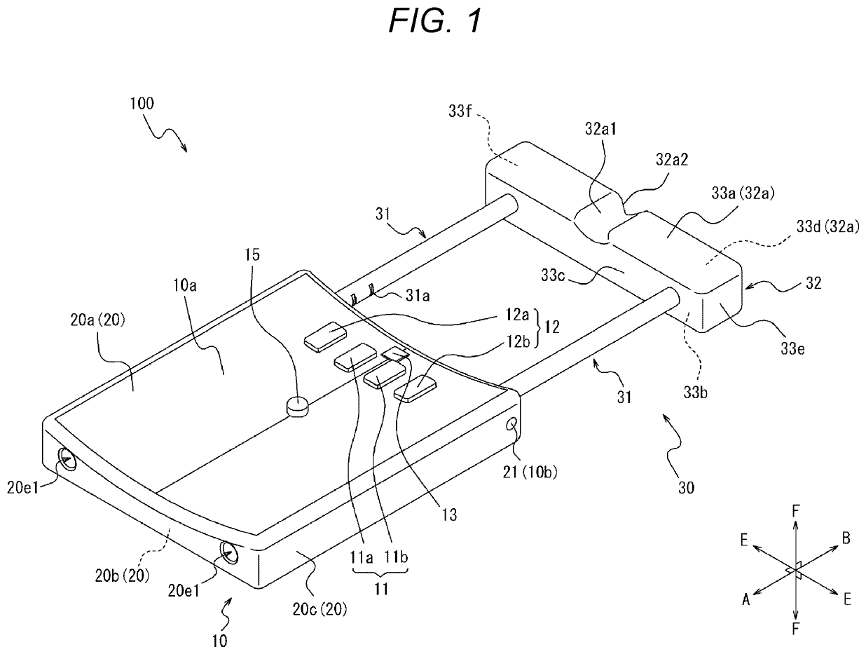

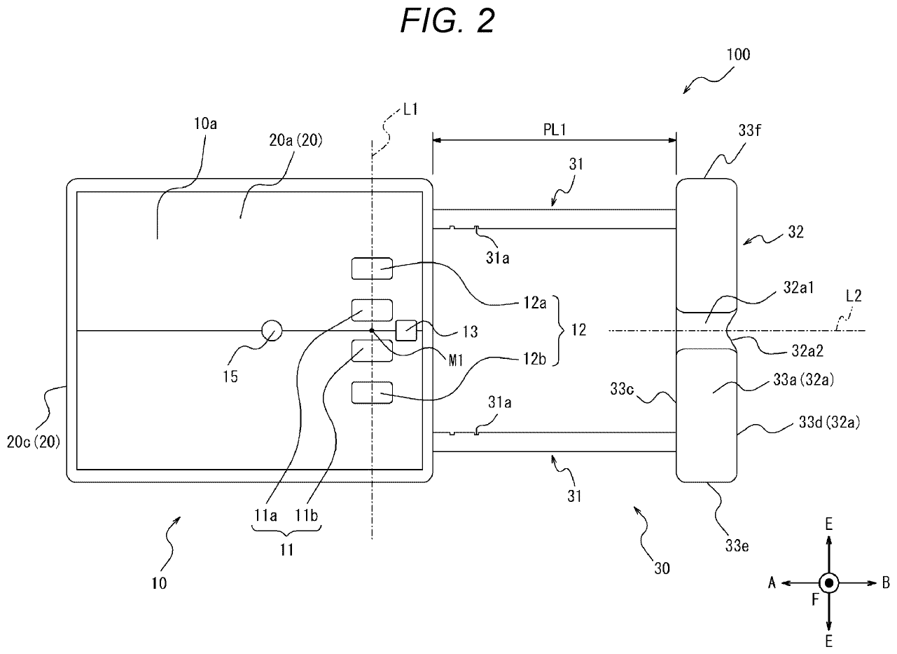

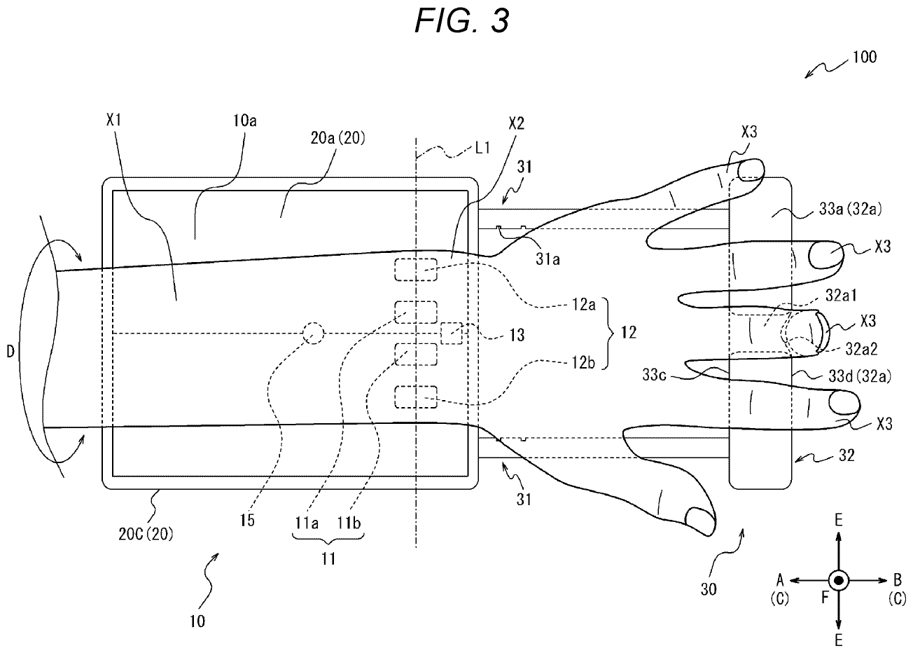

FIG. 1 is a perspective view of a biological information detection device 100 as an embodiment of a biological information detection device according to the present disclosure. FIGS. 2 and 3 are top views of the biological information detection device 100. FIG. 2 illustrates only the biological information detection device 100, and FIG. 3 illustrates an arm and a hand of a user who uses the biological information detection device 100 in addition to the biological information detection device 100.

The biological information detection device 100 is configured to be able to detect biological impedance from a forearm X1 or a wrist X2 of the user. The biological information detection device 100 can measure a water content of the forearm X1 or the wrist X2 of the user by detecting the biological impedance from the forearm X1 or the wrist X2 of the user. It is known that, when a cardiac function of a living body is deteriorated, edema in which moisture accumulates in or under skin occurs. The biological information detection device 100 can measure the water content of the forearm X1 or the wrist X2 of the user such as a heart-disease patient, thereby detecting deterioration in cardiac function of the user in its early stages.

As illustrated in FIGS. 1 to 3, the biological information detection device 100 includes a main body 10 and a finger support 30.

The main body 10 includes an arm placement surface 10a on which the forearm X1 or the wrist X2 of the user can be placed. The arm placement surface 10a may be configured to be able to place both the forearm X1 and the wrist X2 of the user. The main body 10 includes a pair of arm electrodes 11 that is exposed on the arm placement surface 10a and can detect the biological impedance from the forearm X1 or the wrist X2 of the user. Although it will be described later in detail, the arm placement surface 10a of the present embodiment as an example includes a surface curved in a concave shape (circular arc shape) so as to be rather easily fit a surface of the arm. With this configuration, the pair of arm electrodes 11 can easily adhere to a skin surface of the user.

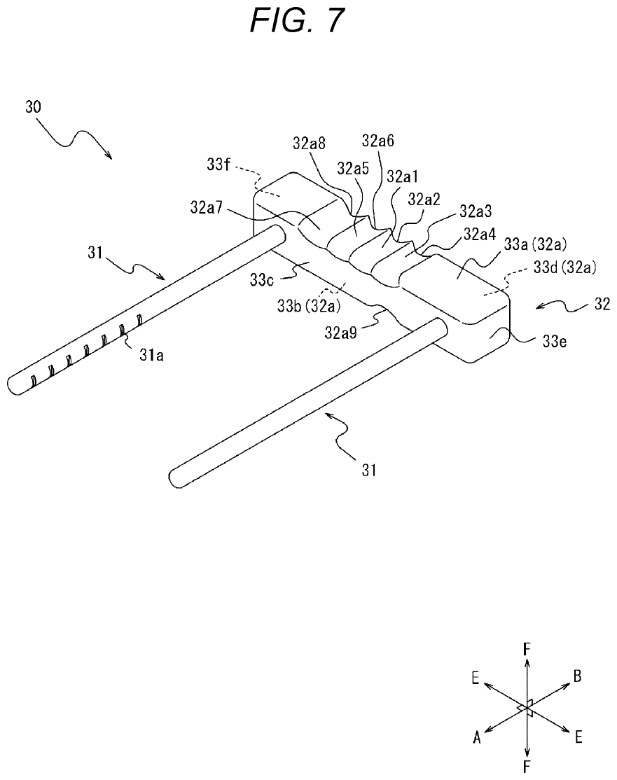

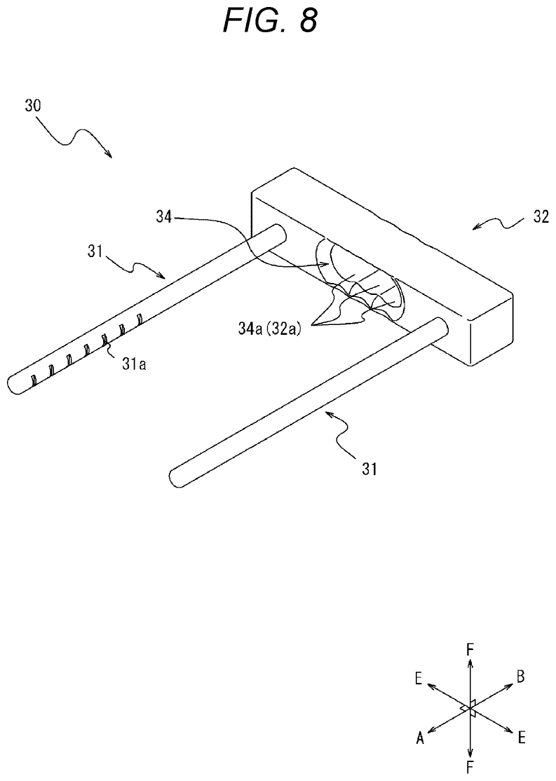

The finger support 30 is configured to be able to support a finger X3 of the user in a state in which the forearm X1 or the wrist X2 of the user is placed on the arm placement surface 10a. Although it will be described later in detail, as illustrated in FIG. 3, the finger support 30 of the present embodiment is configured to be able to support an index finger, a middle finger, a ring finger, and a little finger as the fingers X3 of the user. The finger X3 supported by the finger support 30 is not limited, for example, to the index finger, the middle finger, the ring finger, and the little finger of the present embodiment (refer to FIGS. 7 and 8).

In a top view of the biological information detection device 100 (hereinafter, referred to as a “top view of the biological information detection device 100”) as seen from a side of the arm placement surface 10a, the finger support 30 is attached to the main body 10 so as to be movable in an approaching direction A approaching the main body 10 and a separating direction B separating from the main body 10 opposite to the approaching direction A.

The biological information detection device 100 is used as follows.

The user places his/her forearm X1 or wrist X2 on the arm placement surface 10a of the main body 10 so that the pair of arm electrodes 11 comes into contact with an optimum position of the forearm X1 or the wrist X2. At that time, as illustrated in FIG. 3, the user places the forearm X1 or the wrist X2 on the arm placement surface 10a of the main body 10 so that a length direction C of his/her arm is in the approaching direction A and the separating direction B. When the forearm X1 or the wrist X2 of the user is placed on the arm placement surface 10a of the main body 10, a weight of the forearm X1 or the wrist X2 is applied on the arm placement surface 10a of the main body 10. Next, the user moves the finger support 30 in the approaching direction A and the separating direction B with respect to the main body 10 to adjust the position in which the finger support 30 supports the fingers X3 (in the present embodiment, the index finger, the middle finger, the ring finger, and the little finger of the user). With this configuration, the user can adjust, for example, the position of the finger support 30 according to a length of the hand, a length of the forearm X1 and the like of the user. Next, in this state, the user measures the biological impedance by the biological information detection device 100. A measurement time can be, for example, few minutes such as two or three minutes. During this time, the user keeps a rest state. The number of times of measurement per day may be, for example, one or two, or three or more. Although it will be described later in detail, circular arc grooves 32a1 and 32a2 in which the middle finger of the user can be placed is disposed with the finger support 30 of the present embodiment as an example. By disposing such circular arc grooves 32a1 and 32a2, the circular arc grooves 32a1 and 32a2 can be used as a mark for arranging the center of the hand of the user on the finger support 30.

The user memorizes the position of the finger support 30 adjusted as described above with respect to the main body 10. The user also memorizes a position and a posture of the finger X3 supported by the finger support 30. As an example, the user memorizes a position of a scale as an identification unit 31a disposed in a length adjustment unit 31 to be described later of the present embodiment. With this configuration, the user can adjust the position of the finger support 30 with respect to the main body 10 in the same position as that in current measurement also in next measurement. In the next measurement also, the user can cause the finger support 30 to support the finger X3 in the position and posture similar to those in the current measurement. As a result, the biological information detection device 100 can rather easily bring the pair of arm electrodes 11 into contact with the same position of the forearm X1 or the wrist X2 as in the current measurement even in the next measurement.

As described above, according to biological information detection device 100, the position of the finger support 30 can be set according to the length of the hand, the length of the forearm X1 and the like of the user. According to the biological information detection device 100, by setting the position of the finger support 30 to the above-described setting position at the time of each measurement, it is possible to suppress a variation in position in which the pair of arm electrodes 11 comes into contact with the forearm X1 or the wrist X2 at the time of each measurement. Therefore, the biological impedance can be accurately detected only from the forearm X1 or the wrist X2 of one arm of the user placed on the arm placement surface 10a. The biological information detection device 100 can measure the biological impedance when the user places his/her forearm X1 or wrist X2 on the arm placement surface 10a by its own weight for a predetermined time in a rest state. Therefore, biological information detection device 100 can periodically, easily, and accurately measure the biological impedance in a relatively short time without being worn on arm of the user.

In this manner, by periodically detecting the biological impedance in the same position of the forearm X1 or the wrist X2 of the user by using the biological information detection device 100, it is possible to accurately monitor a change in water content of the forearm X1 or the wrist X2 of the user, which makes it possible to detect deterioration in cardiac function of the user in its early stages.

Hereinafter, the biological information detection device 100 of the present embodiment will be described in further detail with reference to FIGS. 1 to 6.

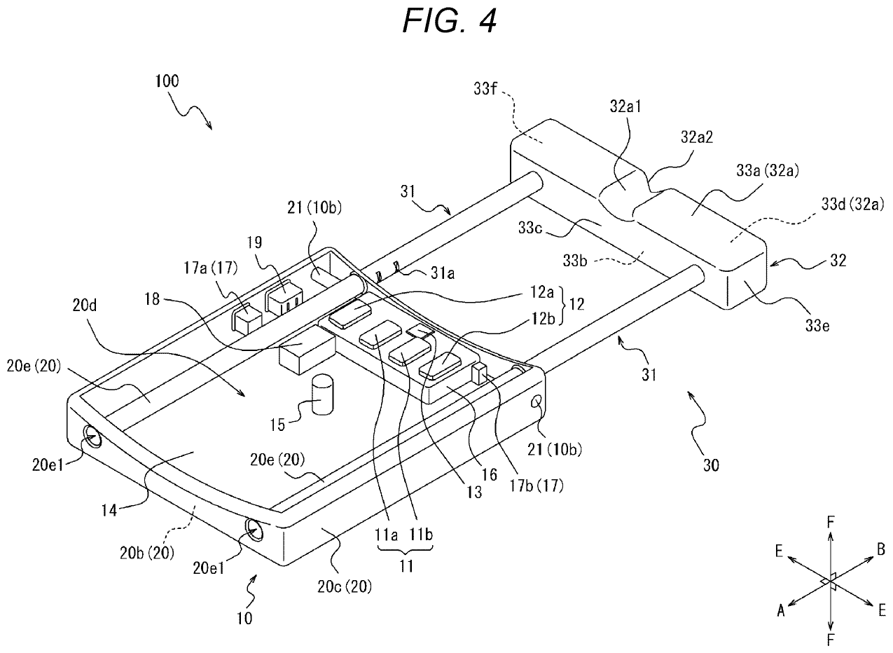

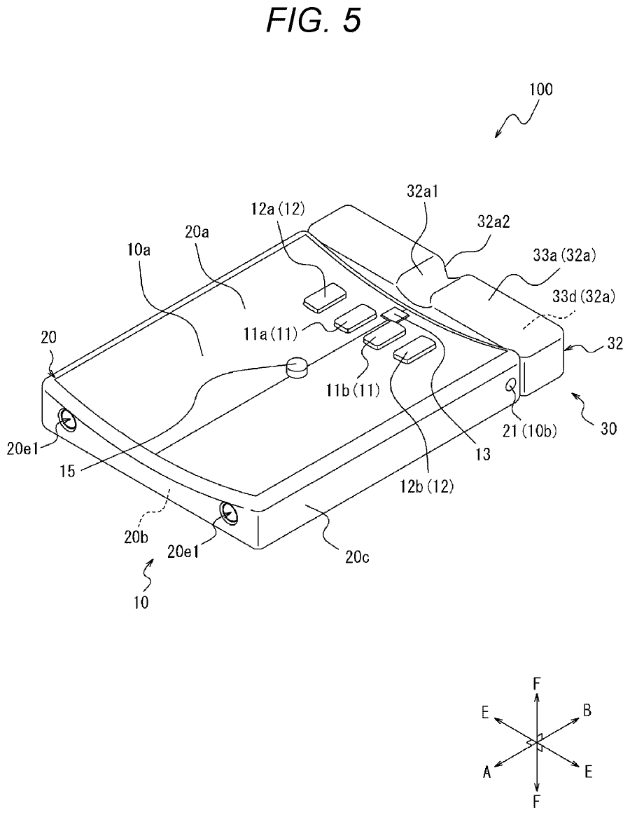

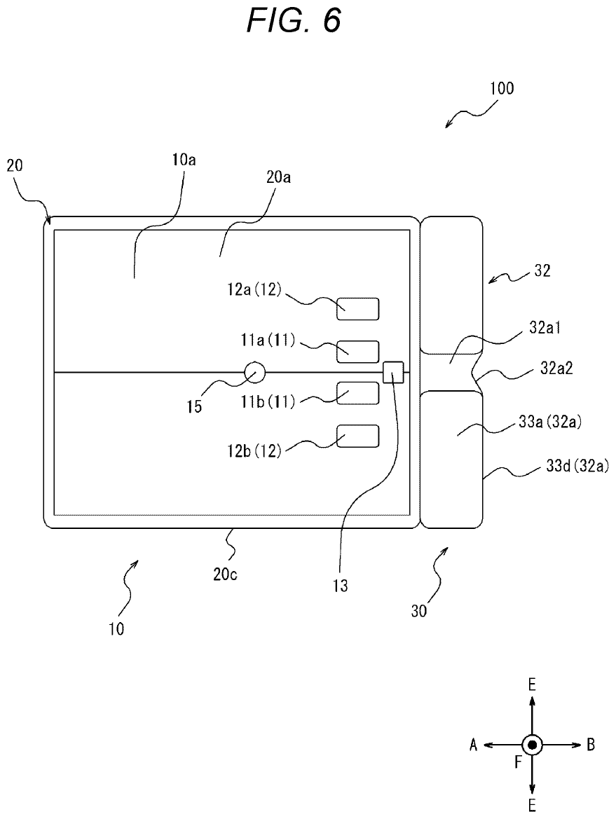

FIG. 4 is a diagram illustrating the inside of the main body 10 of the biological information detection device 100 of the present embodiment in detail. For convenience of description, FIG. 4 illustrates a state in which an upper plate 20a forming the arm placement surface 10a of the main body 10 is omitted. FIG. 5 is a perspective view of the biological information detection device 100. FIG. 1 illustrates a state in which the finger support 30 is pulled out in the separating direction B with respect to the main body 10, whereas FIG. 5 illustrates a state before the finger support 30 is pulled out in the separating direction B with respect to the main body 10. More specifically, the finger support 30 illustrated in FIG. 5 is illustrated in a state of being the closest to the main body 10 in the approaching direction A. Hereinafter, for convenience of description, the state of the biological information detection device 100 illustrated in FIGS. 1 to 4 in which the finger support 30 is pulled out in the separating direction B with respect to the main body 10 is sometimes simply referred to as a “use state of the biological information detection device 100”. The state of the biological information detection device 100 illustrated in FIG. 5 in which the finger support 30 is the closest to the main body 10 in the approaching direction A is sometimes simply referred to as a “stored state of the biological information detection device 100”. FIG. 6 is a top view of the biological information detection device 100 in the stored state.

As described above, the biological information detection device 100 of the present embodiment includes the main body 10 and the finger support 30.

### Main Body 10

As illustrated in FIGS. 1 to 6, the main body 10 of the present embodiment can include two pairs of arm electrodes 11 and 12, a pulse sensor 13, a weight detection unit 14, an activation switch 15, a control unit 16, a communication unit 17, a rechargeable battery 18, a power supply connector unit 19, a housing 20, and a screw member 21.

### Two Pairs of Arm Electrodes 11 and 12

The two pairs of arm electrodes 11 and 12 are exposed on the arm placement surface 10a.

One pair of arm electrodes 11 out of the two pairs of arm electrodes 11 and 12 comes into contact with the forearm X1 or the wrist X2 of the user, and is configured to be able to detect the biological impedance. The other pair of arm electrodes 12 out of the two pairs of arm electrodes 11 and 12 may come into contact with the forearm X1 or the wrist X2 of the user, and may be configured to be able to detect the biological impedance, for example, similarly to one pair of arm electrodes 11. The other pair of arm electrodes 12 may be used, for example, as application electrodes that come into contact with the forearm X1 or the wrist X2 of the user and apply a current to the forearm X1 or the wrist X2. Furthermore, the other pair of arm electrodes 12 may be used, for example, to correct a detection value of the biological impedance by one pair of arm electrodes 11. As described above, the other pair of arm electrodes 12 may be used to improve detection accuracy of the biological impedance by one pair of arm electrodes 11. It is possible that the main body 10 does not include the pair of arm electrodes 12. Hereinafter, for convenience of description, the pair of arm electrodes 11 of the present embodiment will be referred to as “a pair of arm detection electrodes 11”, and the pair of arm electrodes 12 of the present embodiment will be referred to as “a pair of arm auxiliary electrodes 12”.

In top views of the biological information detection device 100 (refer to FIGS. 2, 3, and 6), the pair of arm detection electrodes 11 is arranged side by side in a direction substantially orthogonal to the approaching direction A and the separating direction B. With this configuration, two electrodes 11a and 11b forming the pair of arm detection electrodes 11 can be brought into contact with substantially the same positions in the length direction C of the arm of the user and different positions in a circumferential direction D of the arm of the user. As a result, the pair of arm detection electrodes 11 can detect the biological impedance in one cross section of the arm of the user substantially orthogonal to the length direction C of the arm. Therefore, it becomes rather easy to monitor the change in water content of the arm of the user in association with a change in outer diameter of the arm due to edema and the like.

Hereinafter, for convenience of description, in the top views of the biological information detection device 100 (refer to FIGS. 2, 3, and 6), a direction substantially orthogonal to the approaching direction A and the separating direction B in which the pair of arm detection electrodes 11 is arranged side by side is referred to as an “electrode arrangement direction E”. In the present embodiment, electrodes 12a and 12b forming the pair of arm auxiliary electrodes 12 are also arranged side by side in the electrode arrangement direction E. More specifically, in the present embodiment, the electrodes 11a and 11b of the pair of arm detection electrodes 11 and the electrodes 12a and 12b of the pair of arm auxiliary electrodes 12 are arranged in a line in the electrode arrangement direction E. Here, the fact that the electrodes 11a and 11b of the pair of arm detection electrodes 11 and the electrodes 12a and 12b of the pair of arm auxiliary electrodes 12 are “arranged in a line in the electrode arrangement direction E” means that there is at least one imaginary straight line (for example, an imaginary straight line L1 in FIGS. 2 and 3) that passes through the four electrodes 11a, 11b, 12a, and 12b exposed on the arm placement surface 10a and is parallel to the electrode arrangement direction E in the top view of the biological information detection device 100. With this configuration, it is possible to detect the biological impedance more accurately in one cross section of the arm of the user substantially orthogonal to the length direction C of the arm. In particular, from the viewpoint of further improving the detection accuracy of the biological impedance, it is preferable to match the center positions of the four electrodes 11a, 11b, 12a, and 12b in the approaching direction A and the separating direction B.

Each of the electrodes 11a and 11b of the pair of arm detection electrodes 11 and the electrodes 12a and 12b of the pair of arm auxiliary electrodes 12 can be, for example, a dry electrode made of rubber, metal, and the like.

### Pulse Sensor 13

The pulse sensor 13 is configured to be able to acquire pulse information from the forearm X1 or the wrist X2 of the user placed on the arm placement surface 10a. The configuration of the pulse sensor 13 is not particularly limited, but may be, for example, a so-called PPG sensor using photoplethysmography. Since the main body 10 includes the pulse sensor 13, it is possible to monitor the pulse of the user in addition to the water content of the user. Therefore, the deterioration in cardiac function of the user can be considered from the viewpoint of variation in pulse in addition to the viewpoint of variation in water content.

In the present embodiment, in the top views (refer to FIGS. 2, 3, and 6) of the biological information detection device 100, the pulse sensor 13 is arranged in a position between the pair of arm detection electrodes 11 in the electrode arrangement direction E. More specifically, the pulse sensor 13 of the present embodiment is arranged in a position between the two electrodes 11a and 11b of the pair of arm detection electrodes 11 in the electrode arrangement direction E in the top views (refer to FIGS. 2, 3, and 6) of the biological information detection device 100. With this configuration, the pulse sensor 13 can be arranged closer to the forearm X1 or the wrist X2 of the user placed on the arm placement surface 10a. Therefore, detection accuracy of the pulse by the pulse sensor 13 can be improved.

Although the pulse sensor 13 of the present embodiment is arranged in a position in the separating direction B with respect to the pair of arm detection electrodes 11, the configuration is not limited to this. For example, the pulse sensor 13 may be arranged in a position in the approaching direction A with respect to the pair of arm detection electrodes 11. Note that, it is preferable that the pulse sensor 13 is close to a blood vessel of the arm of the user. Therefore, the pulse sensor 13 is preferably arranged in the vicinity of a position in which a fat amount is relatively small in the arm of the user. Therefore, the pulse sensor 13 is preferably arranged in a position in the separating direction B on the wrist X2 side with respect to the pair of arm detection electrodes 11.

### Weight Detection Unit 14

The weight detection unit 14 is configured to be able to detect weight information of the forearm X1 or the wrist X2 of the user placed on the arm placement surface 10a. The configuration of the weight detection unit 14 is not particularly limited, but this may be formed of, for example, a load cell.

When the cardiac function of the user is deteriorated and the water content in the forearm X1 or the wrist X2 increases, the weight of the forearm X1 or the wrist X2 also increases. That is, since the main body 10 includes the weight detection unit 14, the change in water content of the user can also be confirmed from a weight change. Even in a case where the pair of arm detection electrodes 11 erroneously detects the biological impedance, it is possible to determine the erroneous detection of the pair of arm detection electrodes 11 on the basis of a detection value of the weight detection unit 14.

### Activation Switch 15

The activation switch 15 is exposed on the arm placement surface 10a. The activation switch 15 is configured to be able to be pressed in a direction orthogonal to the arm placement surface 10a. When the activation switch 15 is pressed, the biological information detection device 100 starts a measurement operation of the biological impedance. More specifically, when the activation switch 15 is pressed, the control unit 16 causes the pair of arm detection electrodes 11 to start detecting the biological impedance.

The activation switch 15 of the present embodiment is arranged in a position between the pair of arm detection electrodes 11 in the electrode arrangement direction E in the top views (refer to FIGS. 2, 3, and 6) of the biological information detection device 100. With this configuration, the activation switch 15 is pressed by the forearm X1 or the wrist X2 of the user along with the operation of placing the forearm X1 or the wrist X2 of the user on the arm placement surface 10a. Therefore, the user can start the measurement operation of the biological impedance by the biological information detection device 100 only by executing the operation of placing his/her forearm X1 or wrist X2 on the arm placement surface 10a.

Conversely, the activation switch 15 may return to the state before being automatically pressed when the measurement of the biological impedance by the biological information detection device 100 ends and the user lifts and separates his/her forearm X1 or wrist X2 from the arm placement surface 10a. With this configuration, when the user lifts his/her forearm X1 or wrist X2 from the arm placement surface 10a, the activation switch 15 can be automatically turned off.

Furthermore, although the activation switch 15 of the present embodiment is arranged in a position in the approaching direction A with respect to the pair of arm detection electrodes 11, the configuration is not limited to this. The activation switch 15 may be arranged in a position in the separating direction B with respect to the pair of arm detection electrodes 11. Note that, in relation to a preferable arrangement position of the pulse sensor 13 described above, the activation switch 15 is preferably arranged in a position in the approaching direction A on an elbow side with respect to the pair of arm detection electrodes 11.

As described above, although the activation switch 15 of the present embodiment is configured to be exposed on the arm placement surface 10a of the main body 10, the configuration is not limited to this. The activation switch 15 may be exposed in a position other than the arm placement surface 10a of the main body 10. In such a case, the user may execute an operation of operating the activation switch 15 separately from the operation of placing his/her forearm X1 or wrist X2 on the arm placement surface 10a. The activation switch 15 may be disposed in the finger support 30.

As described above, the activation switch 15 of the present embodiment is configured to be pressed in the direction orthogonal to the arm placement surface 10a and physically moves, but the configuration is not limited to this. The activation switch 15 may be, for example, a touch sensor that can detect the contact of the forearm X1 of the user.

### 72 Control Unit 16

The control unit 16 executes an operation instruction and the like to each unit of the biological information detection device 100. The control unit 16 includes a processor such as a central processing unit (CPU) and an microprocessor unit (MPU). More specifically, the control unit 16 of the present embodiment includes a storage unit such as a read only memory (ROM) and a random access memory (RAM). The storage unit may store, for example, various programs executed by the control unit 16. The biological information detection device 100 may include a storage unit separately from the control unit 16.

The control unit 16 is connected to the activation switch 15. Therefore, when the activation switch 15 is pressed, the control unit 16 instructs each unit of the biological information detection device 100 to start the measurement operation of the biological impedance by the pair of arm detection electrodes 11. Furthermore, when the activation switch 15 is pressed, the control unit 16 of the present embodiment instructs each unit of the biological information detection device 100 including the pulse sensor 13 to cause the pulse sensor 13 to start pulse measurement. Furthermore, when the activation switch 15 is pressed, the control unit 16 of the present embodiment instructs each unit of the biological information detection device 100 including the weight detection unit 14 to cause the weight detection unit 14 to start weight measurement.

The control unit 16 is connected to a changeover switch. The changeover switch switches the connection to the control unit 16 between the rechargeable battery 18 such as, for example, a lithium ion battery and the power supply connector unit 19, thereby enabling power supply to the control unit 16 from any of the rechargeable battery 18 and an external power supply such as a commercial power supply connected to the power supply connector unit 19.

Furthermore, the control unit 16 may execute processing of calculating the water content from the biological impedance detected by the pair of arm detection electrodes 11. The control unit 16 may transmit the calculated water content to an external device such as a smartphone of the user, a server of a medical institution, or a cloud server via the communication unit 17. Note that, the control unit 16 may transmit the detection value of the biological impedance to the external device without executing the processing of calculating the water content from the biological impedance detected by the pair of arm detection electrodes 11. That is, the processing of calculating the water content from the biological impedance detected by the pair of arm detection electrodes 11 may be executed by the external device.

### Communication Unit 17

The communication unit 17 includes at least one of a wireless communication module and a wired communication module. The wireless communication module can be, for example, a communication module compatible with a communication standard such as a wireless local area network (LAN), Bluetooth®, or near field communication (NFC). The wired communication module may be, for example, a wired LAN communication module and the like. As a result, the biological information detection device 100 can perform wireless communication or wired communication with a communication terminal such as a smartphone or an external device such as a server via the communication unit 17. As illustrated in FIG. 4, the communication unit 17 of the present embodiment includes a wired communication module and includes a connection port 17a to which a communication cable can be connected. Furthermore, the communication unit 17 of the present embodiment includes a wireless communication module including an antenna 17b in addition to the wired communication module. Note that, the communication unit 17 may include only one of the wired communication module and the wireless communication module.

### Rechargeable Battery 18 and Power Supply Connector Unit 19

The rechargeable battery 18 can supply power to each unit of the biological information detection device 100. By connecting the external power supply to the power supply connector unit 19, the external power supply can supply power to each unit of the biological information detection device 100. The biological information detection device 100 according to the present embodiment includes both the rechargeable battery 18 and power supply connector unit 19, but the configuration is not limited to this. The biological information detection device 100 may be configured to include only one of the rechargeable battery 18 and power supply connector unit 19.

### Housing 20

The housing 20 is an exterior member of the main body 10. The housing 20 of the present embodiment can be, for example, a box made of resin having a rectangular flat shape. The arm placement surface 10a of the main body 10 of the present embodiment is formed of a surface on one side in a thickness direction of the housing 20, which is a thickness direction F of the main body 10. The thickness direction F of the main body 10 in the present embodiment is a direction orthogonal to the approaching direction A, the separating direction B, and the electrode arrangement direction E.

More specifically, the housing 20 of the present embodiment includes the upper plate 20a and a lower plate 20b arranged to face the upper plate 20a with a gap between the upper plate 20a and the lower plate 20b. The arm placement surface 10a of the main body 10 of the present embodiment includes an outer surface of the upper plate 20a. The gap between the upper plate 20a and the lower plate 20b is an accommodation space 20d (refer to FIG. 4) in which each of the pair of arm detection electrodes 11, the pair of arm auxiliary electrodes 12, the pulse sensor 13, the weight detection unit 14, the activation switch 15, the control unit 16, the communication unit 17, the rechargeable battery 18, and the power supply connector unit 19 described above is partially or entirely accommodated.

The housing 20 of the present embodiment includes a peripheral wall 20c connecting an outer edge of the upper plate 20a and an outer edge of the lower plate 20b. The peripheral wall 20c surrounds the periphery orthogonal to the thickness direction F of the accommodation space 20d described above.

Here, the arm placement surface 10a is formed of a circular arc groove extending from an end in the approaching direction A to an end in the separating direction B of the main body 10. That is, in the present embodiment, the outer surface of the upper plate 20a is formed of a circular arc groove extending from an end in the approaching direction A to an end in the separating direction B. The circular arc groove serving as the arm placement surface 10a is concave in the thickness direction F of the main body 10. More specifically, the circular arc groove as the arm placement surface 10a is concave in a depth direction orthogonal to a paper surface in FIGS. 2, 3, and 6 illustrating the top views of the biological information detection device 100. With this configuration, the forearm X1 or the wrist X2 of the user can be rather easily positioned in the vicinity of a groove bottom of the circular arc groove serving as the arm placement surface 10a. Therefore, the position of the forearm X1 or the wrist X2 of the user placed on the arm placement surface 10a in the electrode arrangement direction E easily becomes stable. As a result, it is possible to further suppress the variation in position in which the pair of arm detection electrodes 11 comes into contact with the forearm X1 or the wrist X2 at the time of each measurement. The pair of arm detection electrodes 11 is arranged on both sides across the groove bottom of the circular arc groove as the arm placement surface 10a in the electrode arrangement direction E. With this configuration, in a state in which the forearm X1 or the wrist X2 of the user is positioned in the vicinity of the groove bottom of the circular arc groove as the arm placement surface 10a, the electrodes 11a and 11b of the pair of arm detection electrodes 11 can more reliably come into contact with different positions in the circumferential direction D of the forearm X1 or the wrist X2. For the similar reason, the arrangement positions of the pair of arm auxiliary electrodes 12 are also arranged on both sides across the groove bottom of the circular arc groove as the arm placement surface 10a in the electrode arrangement direction E. The arm placement surface 10a of the present embodiment is formed only of the circular arc groove described above, but the configuration is not limited to this. For example, the arm placement surface 10a may be configured to include, in addition to the circular arc groove described above, a flat surface portion formed of a flat surface substantially orthogonal to the thickness direction F of the main body 10, the surface continuous with an edge of the circular arc groove on one side or both sides in the electrode arrangement direction E of the circular arc groove.

In the main body 10 of the present embodiment, a surface located on the side opposite to the arm placement surface 10a is formed of a flat surface. That is, in the present embodiment, an outer surface of the lower plate 20b is formed of a flat surface. The surface located on the side opposite to the arm placement surface 10a is a surface placed on a horizontal support surface. Therefore, the surface located on the side opposite to the arm placement surface 10a is preferably formed of a flat surface so that the posture of the main body 10 placed on the horizontal support surface becomes relatively stable.

Furthermore, a plurality of through holes is formed on the upper plate 20a of the housing 20. Each of the pair of arm detection electrodes 11, the pair of arm auxiliary electrodes 12, the pulse sensor 13, and the activation switch 15 described above is accommodated in the accommodation space 20d in such a manner that a part of the pair of arm detection electrodes 11, the pair of arm auxiliary electrodes 12, the pulse sensor 13, and the activation switch 15 protrudes from the arm placement surface 10a through the through hole of the upper plate 20a to be exposed.

As illustrated in FIG. 4, the connection port 17a of the communication unit 17 and the power supply connector unit 19 of the present embodiment are disposed on the peripheral wall 20c. Note that, the connection port 17a of the communication unit 17 and the power supply connector unit 19 may be disposed in other positions of the housing 20.

Furthermore, the housing 20 of the present embodiment includes two cylindrical portions 20e extending in the approaching direction A and the separating direction B in the accommodation space 20d. The cylindrical portion 20e is bridged between two side walls facing each other in the approaching direction A and the separating direction B out of the peripheral wall 20c. That is, both ends of the cylindrical portion 20e are continuous to the two side walls facing each other in the approaching direction A and the separating direction B out of the peripheral wall 20c. The cylindrical portion 20e defines an insertion hole 20e1 through which the length adjustment unit 31 to be described later of the finger support 30 is inserted. Both ends of the insertion hole 20e1 are opened without being closed. Note that, the configuration of the insertion hole 20e1 is not limited to the configuration in which both ends are opened. The insertion hole 20e1 is only required to be configured such that the length adjustment unit 31 of the finger support 30 is insertable so as to be movable in the approaching direction A and the separating direction B, and such that at least the end in the separating direction B is opened. That is, the end of the insertion hole 20e1 in the approaching direction A may be closed.

### Screw Member 21

The screw member 21 includes a male screw portion that can be screwed with a female screw portion disposed on an inner surface of a through hole formed on the peripheral wall 20c of the housing 20. The screw member 21 is configured such that a protruding length on the accommodation space 20d side can vary. A through hole through which the screw member 21 can be inserted is formed on a peripheral wall of the cylindrical portion 20e. A distal end of the screw member 21 can enter the insertion hole 20e1 through the through hole formed on the peripheral wall of the cylindrical portion 20e. Although it will be described later in detail, the screw member 21 forms a position fixing unit 10b that restricts the movement of the length adjustment unit 31 of the finger support 30 in the approaching direction A and the separating direction B with respect to the main body 10.

As described above, the main body 10 of the present embodiment includes the pair of arm detection electrodes 11, the pair of arm auxiliary electrodes 12, the pulse sensor 13, the weight detection unit 14, the activation switch 15, the control unit 16, the communication unit 17, the rechargeable battery 18, the power supply connector unit 19, the housing 20, and the screw member 21 described above, but the configuration is not limited to this. The main body 10 may further include a notification unit such as a speaker or a light emitting unit, for example. For example, the notification unit may notify the outside of a state in which the biological impedance cannot be accurately detected by the pair of arm detection electrodes 11. Specifically, for example, in a case where any one of the forearm X1, the wrist X2, and the finger X3 of the user is not placed in an appropriate position, the notification unit may notify the outside of this. For example, the notification unit may notify the outside in a case where any one of the forearm X1, the wrist X2, and the finger X3 of the user moves during the measurement of the biological impedance. Furthermore, for example, in a case where an abnormal detection value is detected as the biological impedance due to sweat and the like, the notification unit may notify the outside of this for the purpose of prompting re-measurement. In addition, the notification unit may notify the outside of the measurement state of the biological impedance, for example, when the measurement of the biological impedance ends.

### Finger Support 30

As illustrated in FIGS. 1 to 6, the finger support 30 of the present embodiment includes the length adjustment unit 31 and a finger placement table 32. The length adjustment unit 31 is attached to the main body 10 so as to be movable in the approaching direction A and the separating direction B. As a result, as illustrated in FIG. 2, the length adjustment unit 31 is configured to be able to adjust a protruding length PL1 from the main body 10. The finger placement table 32 is held by the length adjustment unit 31. The finger placement table 32 includes a finger placement surface 32a on which an inner surface of the finger X3 can be placed.

### Length Adjustment Unit 31

The finger support 30 of the present embodiment includes two length adjustment units 31. The length adjustment unit 31 of the present embodiment is a linear rod-shaped portion that can be inserted in the insertion hole 20e1 of the cylindrical portion 20e of the housing 20 of the main body 10. The two length adjustment units 31 extend substantially parallel to the approaching direction A and the separating direction B. In top views of the biological information detection device 100 (refer to FIGS. 2, 3, and 6), the two length adjustment units 31 are arranged so as to be separated in the electrode arrangement direction E substantially orthogonal to the approaching direction A and the separating direction B.

The length adjustment unit 31 is inserted in the insertion hole 20e1 so as to be movable in the approaching direction A and the separating direction B. By moving the length adjustment unit 31 in the approaching direction A and the separating direction B, the protruding length PL1 (refer to FIG. 2) of the length adjustment unit 31 protruding from the main body 10 in the separating direction B can be adjusted.

The length adjustment unit 31 of the present embodiment is a rod-shaped portion a cross section of which orthogonal to the approaching direction A and the separating direction B has a substantially circular outer shape, but the configuration is not limited to this. The outer shape of the cross section of the length adjustment unit 31 orthogonal to the approaching direction A and the separating direction B may be, for example, a polygonal shape such as a rectangular shape or an oval shape such as an elliptical shape or an oval shape.

The length adjustment unit 31 of the present embodiment is inserted in the insertion hole 20e1 of the main body 10 and is configured to be movable in the insertion hole 20e1, but the configuration is not limited to this. For example, the length adjustment unit 31 may be configured to slide in the approaching direction A and the separating direction B along a guide rail disposed on the outer surface of the housing 20 of the main body 10. In this manner, the attachment configuration of the length adjustment unit 31 to the main body 10 is not limited to the attachment configuration using the insertion hole 20e1 of the present embodiment.

Furthermore, the length adjustment unit 31 of the present embodiment includes the identification unit 31a that enables the protruding length PL1 to be identified from the outside. The identification unit 31a of the present embodiment is a scale indicating the protruding length PL1. The scale as the identification unit 31a of the present embodiment includes a plurality of concave portions arranged in the approaching direction A and the separating direction B. The plurality of concave potions may be arranged in the approaching direction A and the separating direction B at predetermined intervals of, for example, 5 mm. Since the length adjustment unit 31 includes the identification unit 31a, the user can easily externally identify the protruding length PL1 corresponding to the length of the hand, the length of the forearm X1 and the like of the user. Therefore, when measuring the biological impedance by the biological information detection device 100, the user can always easily set the position of the finger support 30 with respect to the main body 10 to the same position.

In the present embodiment, a relative position of the length adjustment unit 31 with respect to the main body 10 in the approaching direction A and the separating direction B can be fixed by the screw member 21 as the position fixing unit 10b of the main body 10. That is, the screw member 21 of the main body 10 can fix the relative position with respect to the main body 10 in the approaching direction A and the separating direction B of the length adjustment unit 31. Specifically, in the present embodiment, the distal end of the screw member 21 is caused to enter the insertion hole 20e1 of the cylindrical portion 20e, and a side surface of the rod-shaped portion as the length adjustment unit 31 is pressed, so that the movement of the length adjustment unit 31 in the approaching direction A and the separating direction B can be restricted, and the position in the approaching direction A and the separating direction B can be fixed.

In the present embodiment, the main body 10 includes the screw member 21 as the position fixing unit 10b, but the configuration is not limited to this. That is, the length adjustment unit 31 may include the position fixing unit that fixes the relative position with respect to the main body 10 in the approaching direction A and the separating direction B. Furthermore, both the main body 10 and the length adjustment unit 31 may include a position fixing unit that fixes the position in the approaching direction A and the separating direction B of the other.

The configuration of the position fixing unit is not limited to the screw member 21 of the present embodiment. The position fixing unit may be formed of, for example, concavo-convex portions disposed in the main body 10 and the length adjustment unit 31 and can be fitted to each other. Examples of such concavo-convex portion include, for example, a convex portion disposed on one of the main body 10 and the length adjustment unit 31 and a concave portion disposed on the other of the main body 10 and the length adjustment unit 31. That is, the configuration of the position fixing unit is not particularly limited as long as this is the configuration capable of fixing the relative positions of the main body 10 and the length adjustment unit 31 in the approaching direction A and the separating direction B.

Furthermore, the length adjustment unit 31 of the present embodiment is configured to be manually movable in the approaching direction A and the separating direction B with respect to the main body 10 by the user, but the configuration is not limited to this. The length adjustment unit 31 may be electrically movable in the approaching direction A and the separating direction B with respect to the main body 10 by, for example, an electric motor disposed in the main body 10 and a movable mechanism operated by the electric motor. The protruding length PL1 of the length adjustment unit 31 may be adjustable by the control unit 16 controlling the electric motor and the movable mechanism described above. Examples of the movable mechanism described above can include, for example, a mechanism including a rack and pinion and the like.

A constituent material of the length adjustment unit 31 is not particularly limited. The length adjustment unit 31 may be made of resin, for example.

### Finger Placement Table 32

The finger placement table 32 of the present embodiment is held at an end of the length adjustment unit 31 in the separating direction B. More specifically, the finger placement table 32 of the present embodiment is bridged between the ends in the separating direction B of the two rod-shaped portions as the two length adjustment units 31. A constituent material of the finger placement table 32 is not particularly limited. The finger placement table 32 may be made of resin, for example, as the length adjustment unit 31.

The finger placement table 32 of the present embodiment has a rectangular parallelepiped outer shape extending in the electrode arrangement direction E. The finger placement surface 32a of the finger placement table 32 of the present embodiment is formed from a surface on the same side as the arm placement surface 10a of the main body 10 to a surface facing the separating direction B.

More specifically, the finger placement table 32 of the present embodiment includes an upper surface 33a being a surface on the same side as the arm placement surface 10a of the main body 10, and a lower surface 33b facing the upper surface 33a. The upper surface 33a and the lower surface 33b of the present embodiment are surfaces extending in a direction substantially orthogonal to the thickness direction F. In the present embodiment, the direction in which the upper surface 33a and the lower surface 33b face each other is the same as the thickness direction F of the main body 10. Furthermore, the finger placement table 32 of the present embodiment includes a first side surface 33c continuous with ends of the upper surface 33a and the lower surface 33b in the approaching direction A, and a second side surface 33d continuous with ends of the upper surface 33a and the lower surface 33b in the separating direction B. Furthermore, the finger placement table 32 of the present embodiment includes a third side surface 33e continuous with one ends of the upper surface 33a and the lower surface 33b in the electrode arrangement direction E, and a fourth side surface 33f continuous with the other ends of the upper surface 33a and the lower surface 33b in the electrode arrangement direction E. The first side surface 33c, the second side surface 33d, the third side surface 33e, and the fourth side surface 33f are surfaces extending in the thickness direction F.

The finger placement surface 32a of the present embodiment is formed of the upper surface 33a and the second side surface 33d of the finger placement table 32. The finger placement surface 32a of the present embodiment includes a first circular arc groove 32a1 extending from the end in the approaching direction A to the end in the separating direction B on the upper surface 33a, and a second circular arc groove 32a2 continuous with the end in the separating direction B of the first circular arc groove 32a1 and extending in the thickness direction F on the second side surface 33d. In a state in which the user places his/her forearm X1 or wrist X2 on the arm placement surface 10a such that the length direction C of the arm is in the approaching direction A and the separating direction B, the user places the inner surface of his/her finger X3 on the finger placement surface 32a of the finger placement table 32. More specifically, the first circular arc groove 32a1 of the finger placement surface 32a of the present embodiment is configured such that the user places a portion between a first joint and a second joint of an inner surface of his/her middle finger. The second circular arc groove 32a2 of the finger placement surface 32a of the present embodiment is configured such that the user places a distal portion from the first joint of the inner surface of his/her middle finger. In this manner, the user hooks his/her middle finger at a corner between the first circular arc groove 32a1 and the second circular arc groove 32a2 by folding his/her middle finger at the first joint. As a result, the user can place the inner surface of his/her middle finger across the first circular arc groove 32a1 and the second circular arc groove 32a2 of the finger placement surface 32a. At that time, the user may place his/her index finger and ring finger on portions on the upper surface 33a on both sides in the electrode arrangement direction E with respect to the first circular arc groove 32a1.

Note that, a method of placing the finger on the finger placement surface 32a of the finger placement table 32 is not limited to the above-described method. For example, the user may place a portion between the second joint and a third joint of the inner surface of the middle finger on the first circular arc groove 32a1. In such a case, a portion between the first joint and the second joint of the inner surface of the middle finger of the user may be placed on the second circular arc groove 32a2.

In the top views (refer to FIGS. 2, 3, and 6) of the biological information detection device 100, the first circular arc groove 32a1 and the second circular arc groove 32a2 of the finger placement surface 32a of the finger placement table 32 of the present embodiment are arranged in a position between the pair of arm detection electrodes 11 of the main body 10 in the electrode arrangement direction E. Therefore, in a state in which the user places his/her forearm X1 or wrist X2 on the arm placement surface 10a such that the length direction C of the arm is in the approaching direction A and the separating direction B, the user can easily place the inner surface of his/her middle finger on the first circular arc groove 32a1 and the second circular arc groove 32a2. By arranging the first circular arc groove 32a1 and the second circular arc groove 32a2 in the above-described position, it is easy for the user to intuitively grasp the above-described method of placing the finger.

In the biological information detection device 100 of the present embodiment, a length of the main body 10 in the electrode arrangement direction E is substantially equal to a length of the finger placement table 32 in the electrode arrangement direction E. A length of the main body 10 in the thickness direction F is also substantially equal to a length of the finger placement table 32 in the thickness direction F. Furthermore, in the thickness direction F, the arm placement surface 10a of the main body 10 is arranged in a position substantially equal to that of the upper surface 33a forming a part of the finger placement surface 32a of the finger placement table 32. Furthermore, in the thickness direction F, the surface on the opposite side of the arm placement surface 10a of the main body 10 is arranged in a position substantially equal to that of the lower surface 33b of the finger placement table 32. Therefore, the biological information detection device 100 of the present embodiment can easily measure the biological impedance even in a state in which the user is lying down, by placing the forearm X1 or the wrist X2 the user on the arm placement surface 10a of the main body 10 and placing the finger X3 on the finger placement surface 32a of the finger placement table 32 in a posture in which the user stretches his/her arm. That is, the biological information detection device 100 of the present embodiment can be easily used, for example, at a bedside.

As illustrated in FIG. 2, in the top view of the biological information detection device 100, the first circular arc groove 32a1 and the second circular arc groove 32a2 of the finger placement surface 32a are arranged in a position intersecting an imaginary straight line L2 that passes through a midpoint M1 of the pair of arm detection electrodes 11 in the electrode arrangement direction E and is parallel to the approaching direction A and the separating direction B.

Note that, the configuration of the finger placement surface 32a of the finger placement table 32 is not limited to the configuration of the present embodiment. FIGS. 7 and 8 are diagrams illustrating a variation of the finger placement surface 32a of the finger placement table 32. The finger placement surface 32a illustrated in FIG. 7 includes a third circular arc groove 32a3 and a fourth circular arc groove 32a4 for the index finger in addition to the first circular arc groove 32a1 and the second circular arc groove 32a2 for the middle finger. Furthermore, the finger placement surface 32a illustrated in FIG. 7 includes a fifth circular arc groove 32a5 and a sixth circular arc groove 32a6 for the ring finger. Furthermore, the finger placement surface 32a illustrated in FIG. 7 includes a seventh circular arc groove 32a7 and an eighth circular arc groove 32a8 for the little finger. The finger placement surface 32a illustrated in FIG. 7 includes a ninth circular arc groove 32a9 on which an inner surface of the thumb is placed in a state in which the fingers are placed on the first circular arc groove 32a1 to the eighth circular arc groove 32a8. That is, the finger placement surface 32a illustrated in FIG. 7 is configured to be grippable by the user. In the example illustrated in FIG. 7, the finger placement surface 32a is formed of the upper surface 33a, the lower surface 33b, and the second side surface 33d of the finger placement table 32. In this manner, the finger placement surface 32a may include a circular arc groove for placing another finger in addition to the first circular arc groove 32a1 and the second circular arc groove 32a2 for the middle finger. Note that, the finger placement surface 32a is not limited to the configuration illustrated in FIG. 7, and may be configured to include only a part of the third circular arc groove 32a3 to the ninth circular arc groove 32a9.

As illustrated in FIG. 8, the finger placement surface 32a may be formed of an inner surface of a through hole 34 penetrating from an end in the approaching direction A to an end in the separating direction B of the finger placement table 32. The through hole 34 illustrated in FIG. 8 is configured to allow insertion of the index finger, the middle finger, and the ring finger of the user. With the index finger, the middle finger, and the ring finger inserted through the through hole 34, the user places the respective fingers of the index finger, the middle finger, and the ring finger on the circular arc groove 34a formed on the inner surface of the through hole 34. In FIG. 8, three circular arc grooves 34a arranged in parallel are formed on each of the surfaces on both sides in the thickness direction F of the inner surface of the through hole 34, but three circular arc grooves 34a may be arranged only on the surface on one side in the thickness direction F on which the inner surface of the finger X3 is placed.

Although the through hole 34 penetrating from the end in the approaching direction A to the end in the separating direction B is formed on the finger placement table 32 illustrated in FIG. 8, the configuration is not limited to this. The finger placement table 32 may include, for example, a concave portion extending from the end in the approaching direction A toward the separating direction B and terminating before reaching the end in the separating direction B instead of the through hole 34. The finger placement surface 32a may be formed of an inner surface of the concave portion of the finger placement table 32.

The through hole 34 illustrated in FIG. 8 may be configured to allow insertion of only the index finger of the user. The through hole 34 may be configured to allow insertion of only two fingers of the middle finger and either the index finger or ring finger of the user.

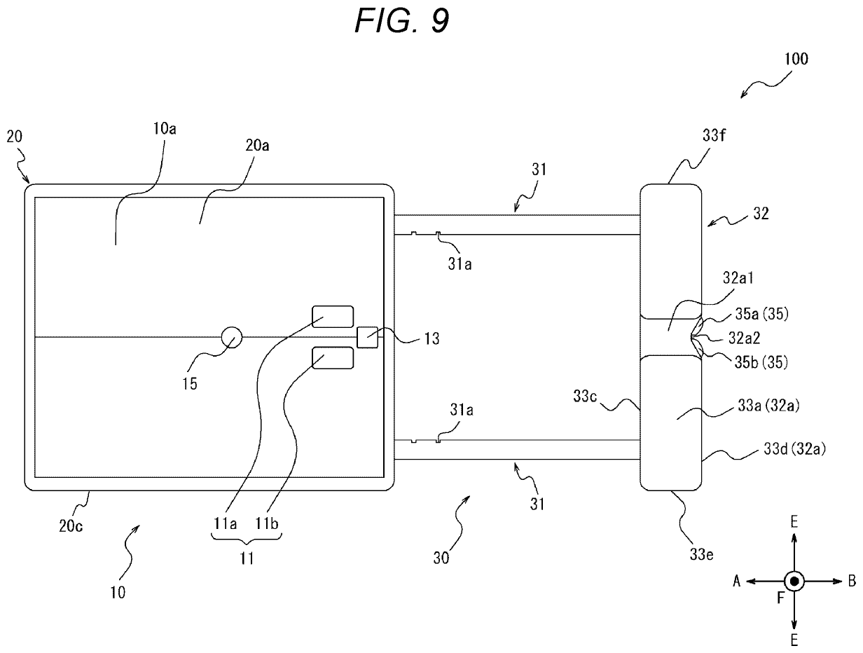

As illustrated in FIG. 9, the finger placement table 32 may include a pair of finger electrodes 35 that is exposed on the finger placement surface 32a and is capable of detecting the biological impedance through the inner surface of the finger X3 (refer to FIG. 3). By disposing such pair of finger electrodes 35, the biological impedance can be detected from the finger X3 in addition to the biological impedance detected by the pair of arm detection electrodes 11 of the main body 10. Therefore, by considering both the biological impedance detected by the pair of arm detection electrodes 11 and the biological impedance detected by the pair of finger electrodes 35, erroneous detection of the biological impedance can be suppressed.

The pair of finger electrodes 35 illustrated in FIG. 9 is arranged side by side in the electrode arrangement direction E in the second circular arc groove 32a2 of the finger placement surface 32a, but the configuration is not limited to this. The pair of finger electrodes 35 may be arranged side by side in the electrode arrangement direction E in the first circular arc groove 32a1 of the finger placement surface 32a. The two electrodes 35a and 35b of the pair of finger electrodes 35 illustrated in FIG. 9 are arranged so as to be in contact with the same finger X3 (refer to FIG. 3), but the configuration is not limited to this. The two electrodes 35a and 35b of the pair of finger electrodes 35 may be arranged so as to be in contact with different fingers X3.

The main body 10 of the biological information detection device 100 illustrated in FIG. 9 does not include the pair of arm auxiliary electrodes 12, but the configuration is not limited to this. Similarly to the configuration illustrated in FIGS. 1 to 6, the main body 10 may further include the pair of arm auxiliary electrodes 12.

As described above, the biological information detection device 100 of the present embodiment includes the main body 10 and the finger support 30. The finger support 30 of the present embodiment includes the length adjustment unit 31 and the finger placement table 32. The biological information detection device 100 of the present embodiment can be downsized by being put into the stored state in which the finger support 30 is pushed in the approaching direction A the most with respect to the main body 10. More specifically, in the present embodiment, the length adjustment unit 31 may be moved in the approaching direction A to a position in which the finger placement table 32 abuts the main body 10. The biological information detection device 100 can be easily carried by being put in this stored state. Therefore, this is portable when going out or taking a long trip. When using the biological information detection device 100, the user pulls out the finger support 30 in the separating direction B to put the same into a use state.

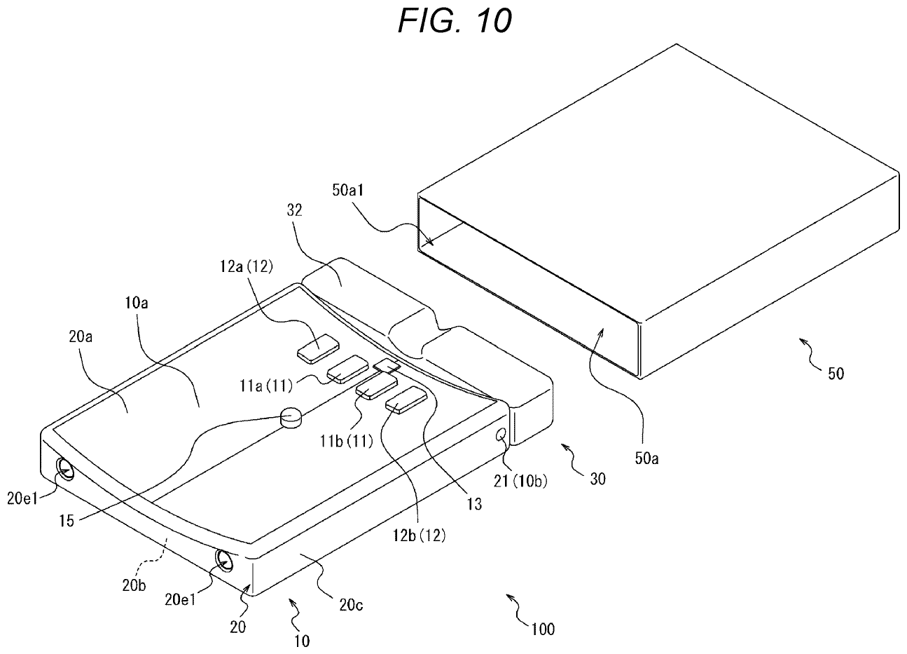

Here, as illustrated in FIG. 10, the biological information detection device 100 may further include an accommodation case 50 capable of accommodating the main body 10 and the finger support 30 in the state in which the finger support 30 is pushed in the approaching direction A the most with respect to the main body 10. The accommodation case 50 illustrated in FIG. 10 has a rectangular box-shaped outer shape capable of accommodating the main body 10 and the finger support 30 in the stored state of the biological information detection device 100 in which the finger support 30 is pushed in the approaching direction A the most with respect to the main body 10. The accommodation case 50 illustrated in FIG. 10 defines an accommodation space 50a that accommodates the main body 10 and the finger support 30. One end side of the accommodation space 50a is opened, and the main body 10 and the finger support 30 are accommodated in the accommodation space 50a through an end opening 50a1.

A shape of the accommodation case 50 is not particularly limited. That is, the outer shape of the accommodation case 50 is not limited to the rectangular box shape illustrated in FIG. 10. The accommodation case 50 may include a lid that closes the end opening 50a1 of the accommodation space 50a in a state in which the main body 10 and the finger support 30 are accommodated in the accommodation space 50a.

The biological information detection device according to the present disclosure is not limited to the specific configurations illustrated in the above-described embodiment and variation, and various changes and modifications can be made without departing from the scope of claims.

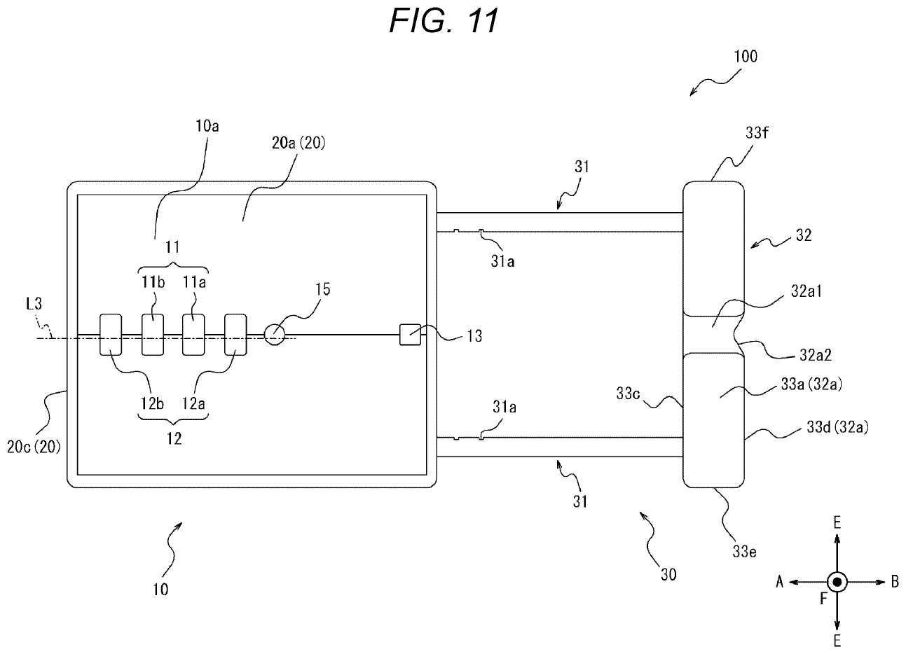

As illustrated in FIGS. 1 to 6, the electrodes 11a and 11b of the pair of arm detection electrodes 11 and the electrodes 12a and 12b of the pair of arm auxiliary electrodes 12 are preferably arranged in a line in the electrode arrangement direction E substantially orthogonal to the approaching direction A and the separating direction B. Note that, the arrangement of the pair of arm detection electrodes 11 and the pair of arm auxiliary electrodes 12 is not limited to this arrangement. FIGS. 11 to 13 are diagrams illustrating a variation of the biological information detection device 100. The arrangement of the pair of arm detection electrodes 11 and the pair of arm auxiliary electrodes 12 may be, for example, the arrangement illustrated in FIGS. 11 to 13.

As illustrated in FIG. 11, the electrodes 11a and 11b of the pair of arm detection electrodes 11 may be arranged side by side in the approaching direction A and the separating direction B. As illustrated in FIG. 11, the electrodes 12a and 12b of the pair of arm auxiliary electrodes 12 may be arranged side by side in the approaching direction A and the separating direction B. Furthermore, as illustrated in FIG. 11, the electrodes 11a and 11b of the pair of arm detection electrodes 11 and the electrodes 12a and 12b of the pair of arm auxiliary electrodes 12 may be arranged in a line in the approaching direction A and the separating direction B. Here, the fact that the electrodes 11a and 11b of the pair of arm detection electrodes 11 and the electrodes 12a and 12b of the pair of arm auxiliary electrodes 12 are “arranged in a line in the approaching direction A and the separating direction B” means that there is at least one imaginary straight line (for example, an imaginary straight line L3 in FIG. 11) that passes through the four electrodes 11a, 11b, 12a, and 12b exposed on the arm placement surface 10a and is parallel to the approaching direction A and the separating direction B in the top view of the biological information detection device 100. The four electrodes 11a, 11b, 12a, and 12b illustrated in FIG. 11 are positioned in the approaching direction A on the side opposite to the finger placement table 32 side with respect to the activation switch 15, but may be positioned in the separating direction B on the finger placement table 32 side with respect to the activation switch 15.

As illustrated in FIG. 11, by arranging the four electrodes 11a, 11b, 12a, and 12b in a line in the approaching direction A and the separating direction B, the measurement can be easily performed on the user whose forearm X1 (refer to FIG. 3) or wrist X2 (refer to FIG. 3) is relatively thin. It becomes easy to secure a separation distance of the four electrodes 11a, 11b, 12a, and 12b in the approaching direction A and the separating direction B. Therefore, it is easy to increase a size of each of the four electrodes 11a, 11b, 12a, and 12b in the approaching direction A and the separating direction B, and in a direction substantially orthogonal to the approaching direction A and the separating direction B (the same direction as the electrode arrangement direction E in FIGS. 1 to 6). It becomes easy to increase the separation distance of the four electrodes 11a, 11b, 12a, and 12b, and measurement accuracy of the biological impedance can be improved.

It is preferable that the four electrodes 11a, 11b, 12a, and 12b illustrated in FIG. 11 are arranged in positions overlapping the groove bottom of the circular arc groove as the arm placement surface 10a in the top view of the biological information detection device 100. With this arrangement, the four electrodes 11a, 11b, 12a, and 12b easily come into contact with the forearm X1 (refer to FIG. 3) or the wrist X2 (refer to FIG. 3) of the user.

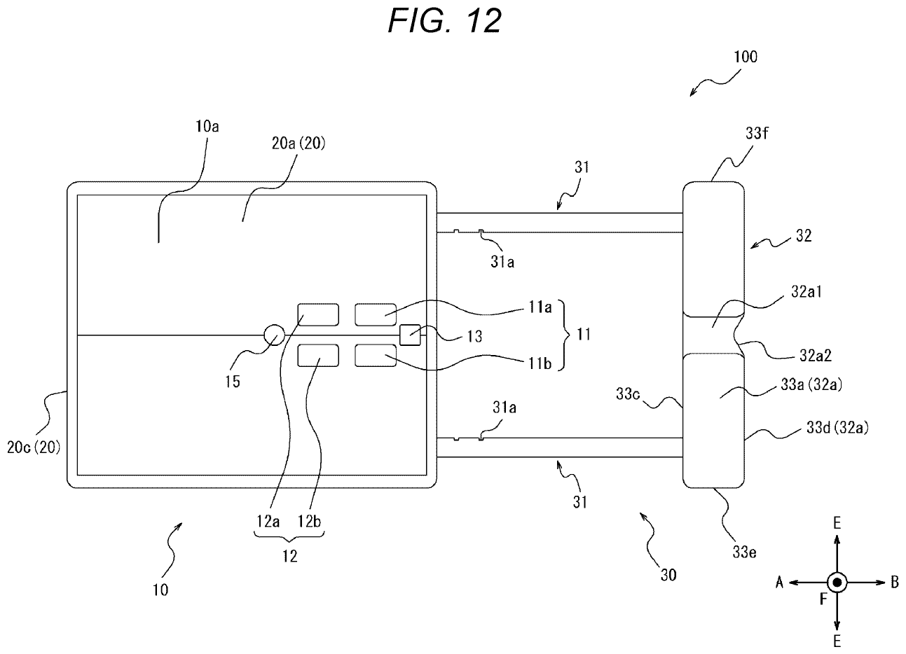

As illustrated in FIG. 12, the pair of arm detection electrodes 11 arranged side by side in the electrode arrangement direction E substantially orthogonal to the approaching direction A and the separating direction B and the pair of arm auxiliary electrodes 12 arranged side by side in the electrode arrangement direction E substantially orthogonal to the approaching direction A and the separating direction B may be arranged in different positions in the approaching direction A and the separating direction B. In FIG. 12, the pair of arm auxiliary electrodes 12 is arranged in positions in the approaching direction A opposite to the finger placement table 32 side with respect to the pair of arm detection electrodes 11. Note that, the pair of arm auxiliary electrodes 12 may be arranged in positions in the separating direction B on the finger placement table 32 side with respect to the pair of arm detection electrodes 11.

Furthermore, the pair of arm detection electrodes 11 arranged side by side in the approaching direction A and the separating direction B, and the pair of arm auxiliary electrodes 12 arranged side by side in the approaching direction A and the separating direction B may be arranged in different positions in the direction substantially orthogonal to the approaching direction A and the separating direction B (the same direction as the electrode arrangement direction E in FIG. 12).

With this arrangement, as compared with the configuration illustrated in FIGS. 1 to 6, an arrangement region in the direction substantially orthogonal to the approaching direction A and the separating direction B of the four electrodes 11a, 11b, 12a, 12b can be reduced. As compared with the configuration illustrated in FIG. 11, an arrangement region in the approaching direction A and the separating direction B of the four electrodes 11a, 11b, 12a, 12b can be reduced. Therefore, for example, the measurement can be easily performed on a user such as a child whose forearm X1 (refer to FIG. 3) and wrist X2 (refer to FIG. 3) are relatively thin and whose arm length is short.

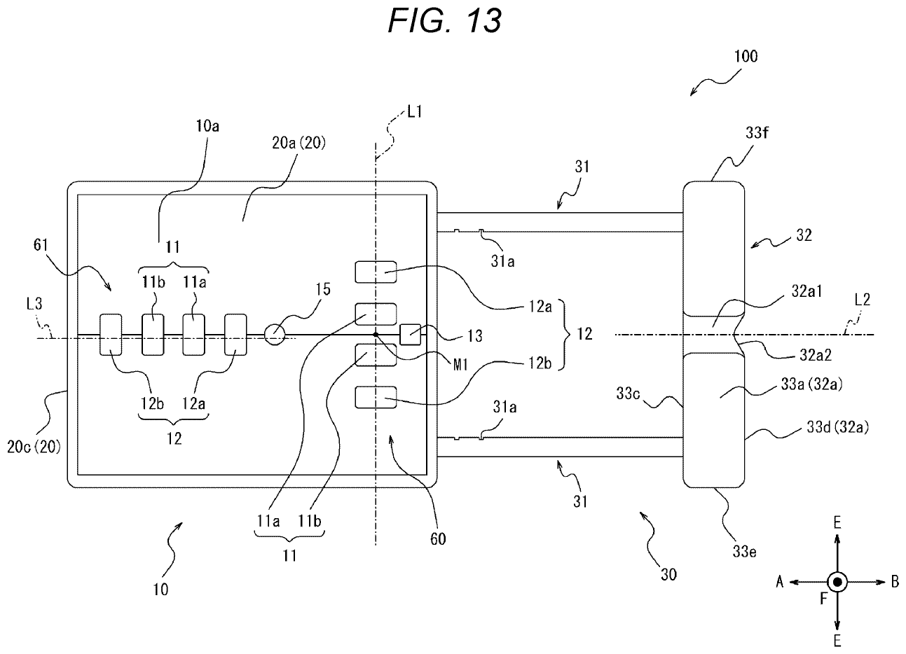

As illustrated in FIG. 13, the biological information detection device 100 may include both the pair of arm detection electrodes 11 arranged side by side in the electrode arrangement direction E substantially orthogonal to the approaching direction A and the separating direction B, and the pair of arm detection electrodes 11 arranged side by side in the approaching direction A and the separating direction B. More specifically, the biological information detection device 100 may include both the pair of arm detection electrodes 11 and the pair of arm auxiliary electrodes 12 arranged in a line in the electrode arrangement direction E substantially orthogonal to the approaching direction A and the separating direction B, and the pair of arm detection electrodes 11 and the pair of arm auxiliary electrodes 12 arranged in a line in the approaching direction A and the separating direction B. Hereinafter, for convenience of description, in FIG. 13, the pair of arm detection electrodes 11 and the pair of arm auxiliary electrodes 12 arranged in a line in the electrode arrangement direction E substantially orthogonal to the approaching direction A and the separating direction B are referred to as a “first electrode set 60”. In FIG. 13, the pair of arm detection electrodes 11 and the pair of arm auxiliary electrodes 12 arranged in a line in the approaching direction A and the separating direction B are referred to as a “second electrode set 61”.

The first electrode set 60 illustrated in FIG. 13 may be arranged in the same position as the pair of arm detection electrodes 11 and the pair of arm auxiliary electrodes 12 illustrated in FIGS. 1 to 6. The second electrode set 61 illustrated in FIG. 13 may be arranged in the same position as the pair of arm detection electrodes 11 and the pair of arm auxiliary electrodes 12 illustrated in FIG. 11.

At least one electrode set of the first electrode set 60 and the second electrode set 61 illustrated in FIG. 13 may be the arrangement of the pair of arm detection electrodes 11 and the pair of arm auxiliary electrodes 12 illustrated in FIG. 12.

In this manner, since the biological information detection device 100 includes both the first electrode set 60 and the second electrode set 61, it is possible to use two systems of detection results of the detection result by the first electrode set 60 and the detection result by the second electrode set 61 as compared with the configuration including only one of them, so that it is possible to improve the detection accuracy. For example, the biological information detection device 100 illustrated in FIG. 13 may measure the biological impedance using the second electrode set 61 after measuring the biological impedance using the first electrode set 60. For example, the biological information detection device 100 may select a measurement value of the biological impedance of any one of both measurement results to output. The biological information detection device 100 may output, for example, the biological impedance corrected on the basis of both the measurement results.

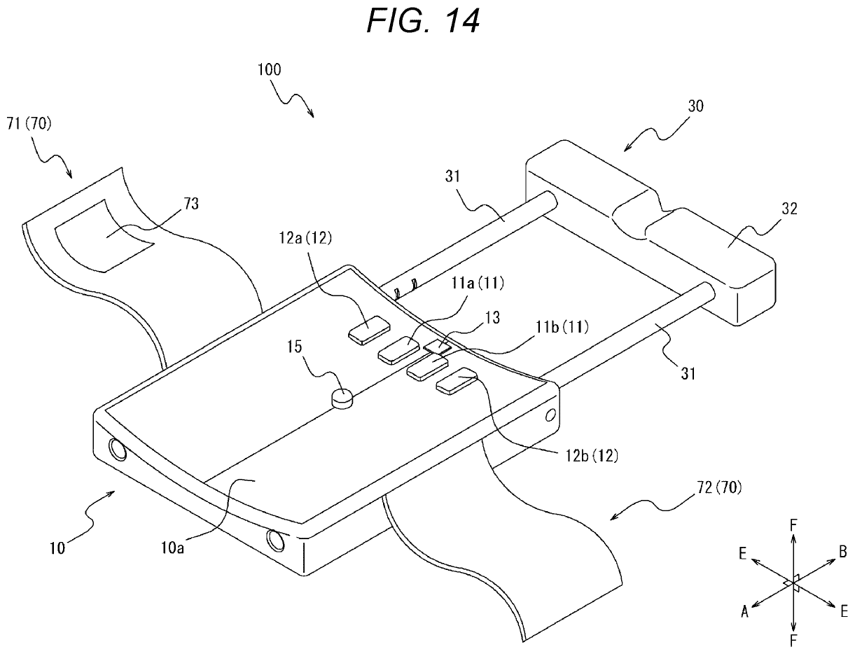

FIG. 14 is a diagram illustrating another variation of the biological information detection device 100. As illustrated in FIG. 14, the biological information detection device 100 may further include a band member 70 attachable to and detachable from the main body 10. The band member 70 is configured to be able to interpose the arm of the user between the same and the arm placement surface 10a of the main body 10 in a state in which the arm of the user is placed on the arm placement surface 10a of the main body 10. That is, the band member 70 can fix the arm of the user on the arm placement surface 10a so as not to separate from the arm placement surface 10a. As a result, a contact state between the pair of arm detection electrodes 11 and the pair of arm auxiliary electrodes 12 and the arm of the user can be made stable. Therefore, the detection accuracy by the biological information detection device 100 can be improved. The detection time by the biological information detection device 100 can be shortened.

As illustrated in FIG. 14, the band member 70 may include, for example, a first band 71 and a second band 72 having flexibility. For example, the first band 71 may be detachably attached to one end of the main body 10 in the electrode arrangement direction E substantially orthogonal to the approaching direction A and the separating direction B. For example, the second band 72 may be detachably attached to the other end of the main body 10 in the electrode arrangement direction E substantially orthogonal to the approaching direction A and the separating direction B. One of the first band 71 and the second band 72 includes a connection unit 73 connectable to the other. The connection unit 73 may be, for example, a hook-and-loop fastener to which the first band 71 and the second band 72 can be attached. The first band 71 and the second band 72 can be connected by the connection unit 73 in a state of covering the arm of the user on the arm placement surface 10a. The first band 71 and the second band 72 are configured to be attachable to and detachable from the main body 10 by, for example, a hook-and-loop fastener and the like.

The configuration of the band member 70 is not limited to the configuration illustrated in FIG. 14. For example, the band member 70 may be configured not to include two portions of the first band 71 and the second band 72. That is, the band member 70 may be, for example, a single elastic band in which one end and the other end are detachably attached to the main body 10. The band member 70 may be, for example, a single endless elastic band that can be attached to and detached from the main body 10.

The detailed description above describes embodiments of a biological information detection device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A biological information detection device configured to detect biological impedance from a forearm or a wrist of a user, the biological information detection device comprising:
a main body including an arm placement surface configured to receive the forearm or the wrist of the user;
a finger support configured to support a finger of the user in a state in which the forearm or the wrist is placed on the arm placement surface;
the main body including a pair of arm electrodes exposed on the arm placement surface and configured to detect the biological impedance from the forearm or the wrist; and
wherein in a top view of the biological information detection device as seen from a side of the arm placement surface, the finger support is attached to the main body so as to be movable in an approaching direction approaching the main body and a separating direction separating from the main body, the separating direction being opposite to the approaching direction.

2. The biological information detection device according to claim 1, wherein
in the top view, the pair of arm electrodes is arranged side by side in a direction substantially orthogonal to the approaching direction and the separating direction.

3. The biological information detection device according to claim 2, wherein
the main body includes a pulse sensor configured to acquire pulse information from the forearm or the wrist placed on the arm placement surface; and
in a case where a direction in which the pair of arm electrodes is arranged in the top view is an electrode arrangement direction, the pulse sensor is arranged in a position between the pair of arm electrodes in the electrode arrangement direction in the top view.

4. The biological information detection device according to claim 2, wherein
the main body includes an activation switch exposed on the arm placement surface; and
in a case where the direction in which the pair of arm electrodes is arranged in the top view is the electrode arrangement direction, the activation switch is arranged in a position between the pair of arm electrodes in the electrode arrangement direction in the top view.

5. The biological information detection device according to claim 1, wherein
the main body includes a weight detection unit configured to detect weight information of the forearm or the wrist placed on the arm placement surface.

6. The biological information detection device according to claim 1, wherein
the arm placement surface includes a circular arc groove extending from an end in the approaching direction to an end in the separating direction of the main body.

7. The biological information detection device according to claim 1, wherein the finger support includes:
a length adjustment unit movably attached to the main body in the approaching direction and the separating direction, the length adjustment unit being configured to adjust a protruding length from the main body; and
a finger placement table held by the length adjustment unit and including a finger placement surface configured to receive an inner surface of the finger.

8. The biological information detection device according to claim 7, wherein
one of the main body and the length adjustment unit includes a position fixing unit configured to fix a relative position of the other of the main body and the length adjustment unit in the approaching direction and the separating direction.

9. The biological information detection device according to claim 7, wherein
the finger placement surface includes a circular arc groove extending from an end in the approaching direction to an end in the separating direction of the finger placement table.

10. The biological information detection device according to claim 7, wherein
the finger placement surface includes an inner surface of a through hole penetrating from an end in the approaching direction to an end in the separating direction of the finger placement table.

11. The biological information detection device according to claim 7, wherein
the finger placement table includes a pair of finger electrodes exposed on the finger placement surface and configured to detect the biological impedance through the inner surface of the finger.

12. A biological information detection device configured to detect biological impedance from a forearm or a wrist of a user, the biological information detection device comprising:
a main body including an arm placement surface on which the forearm or the wrist can be placed;
a pair of arm electrodes exposed on the arm placement surface and configured to detect the biological impedance from the forearm or the wrist;
a pulse sensor placed on the arm placement surface and configured to acquire pulse information from the forearm or the wrist; and
wherein the pulse sensor is placed closer to a peripheral wall of the main body than the pair of arm electrodes so as to position the wrist of the user closer to the pulse sensor than the pair of arm electrodes when placing the forearm or the wrist of the user on the arm placement surface.

13. The biological information detection device according to claim 12, wherein
in the top view, the pair of arm electrodes is arranged side by side.

14. The biological information detection device according to claim 12, further comprising:
an activation switch exposed on the arm placement surface of the main body.

15. The biological information detection device according to claim 12, further comprising:
a weight detection unit on the main body, and wherein the weight detection unit is configured to detect weight information of the forearm or the wrist placed on the arm placement surface.

16. A method for detecting biological impedance from a forearm or a wrist of a user, the method comprising:
receiving the forearm or the wrist of the user on an arm placement surface;
supporting a finger of the user in a state in which the forearm or the wrist is placed on the arm placement surface, wherein in a top view as seen from a side of the arm placement surface, the finger support is attached to a main body so as to be movable in an approaching direction approaching and a separating direction, the separating direction being opposite to the approaching direction; and
detecting the biological impedance from the forearm or the wrist with a pair of arm electrodes exposed on the arm placement surface.

17. The method according to claim 16, further comprising:
in the top view, arranging the pair of arm electrodes side by side in a direction substantially orthogonal to the approaching direction and the separating direction.

18. The method according to claim 17, further comprising:
acquiring pulse information from the forearm or the wrist placed on the arm placement surface from a pulse sensor; and
in a case where a direction in which the pair of arm electrodes is arranged in the top view is an electrode arrangement direction, the pulse sensor is arranged in a position between the pair of arm electrodes in the electrode arrangement direction in the top view.

19. The method according to claim 16, further comprising:
detecting weight information of the forearm or the wrist placed on the arm placement surface with a weight detection unit.

20. The method according to claim 16, wherein the arm placement surface includes a circular arc groove extending from an end in the approaching direction to an end in the separating direction of the main body.
